(19) United States
(12) Patent Application Publication
(10) Pub. No.: US 2015/0342792 A1
(43) Pub. Date: Dec. 3, 2015

(54) DISPOSABLE TOWEL

(71) Applicant: Jorge de la Torre Paniagua, Guatemala (GT)

(72) Inventor: Jorge de la Torre Paniagua, Guatemala (GT)

(21) Appl. No.: 14/727,002

(22) Filed: Jun. 1, 2015

Related U.S. Application Data
(60) Provisional application No. 62/006,789, filed on Jun. 2, 2014.

(51) Int. Cl. A61F 13/471; A61F 13/51; A61F 13/515

(52) U.S. Cl. CPC A61F 13/471; A61F 13/515; A61F 13/52; A61F 2013/4708

(57) ABSTRACT

Embodiments encompass both a disposable and a reusable urine shield towel that includes a stacked layer which encompasses, at least, a waterproof layer sandwiched between a first absorbent layer and a second absorbent layer and further having an opening in an upper portion of the stacked layer to allow for urination therethrough. Embodiments include both reusable and disposable urine shield towels.

### CROSS-REFERENCE TO RELATED APPLICATIONS

This application claims the benefit of priority from Provisional Application Ser. No. 62/006,789 filed on Jun. 2, 2014, which is hereby incorporated by reference in its entirety.

### TECHNICAL FIELD

The following description relates generally to a urine shield (interchangeably referred to as a urine shield towel or a towel), and more specifically to a disposable urine shield.

### BACKGROUND

A problem that can affect men of all ages (both adults and children) when urinating is unwanted urine drops on an upper side of their pants. In addition, many public urinals are designed in such a way that drops of urine and waste or dirty water from the urinal can bounce back to the upper side of the pants when urine falls upon the urinal. Further, unwanted urine drops can fall on a boy's pants when learning proper urination techniques.

Aside from a hygiene problem, when pants are stained with drops of urine or waste water, the current options available are limited to letting the pants dry or to trying to wash the pants in the bathroom, the latter of which is not ideal in a public restroom situation. Further, as urine or waste water drops can leave nasty yellowish stains, a solution that eliminates or at least reduces the chance of such drops from reaching the pants would be desirable.

### SUMMARY

The following presents a simplified description of various exemplary embodiments in accordance with the present invention. Thus this summary is not an extensive overview of all contemplated embodiments in accordance with the present invention or the various aspects of such embodiments. Rather, its purpose is to present some concepts of the present invention in a manner that will allow the more detailed description that is presented later to be more readily understood. Modifications, adaptations or variations of such exemplary embodiments described herein may become apparent to those skilled in the art as such are disclosed. It will be understood that all such modifications, adaptations or variations that rely upon the teachings of the present invention, and through which these teachings have advanced the art, are considered to be within the scope and spirit of the present invention

Exemplary embodiments in accordance with the present invention encompass a disposable urine shield towel (DUST) for male hygienic urination, while other embodiments encompass a reusable urine shield towel (RUST).

Such DUST and RUST embodiments encompass a stacked layer of materials for preventing urine or waste water droplets from reaching a urinating man or boy's pants. In some such embodiments the stacked layer encompasses a first layer, a second layer and a third layer where the second layer is disposed between the first and third layers. Additionally, such embodiments can encompass an opening through an upper portion of such stacked layer to allow for the passage of urine.

In some embodiments the first and third layers can be formed of absorbent material while the second layer can be formed of a non-absorbent material such as a flexible plastic-type material. In some embodiments the second layer material is formed of a natural fabric-type material while in other embodiments such layer is formed of a synthetic fabric-type material.

Each of the layers encompassed in an embodiment of the present invention have an outer surface and an inner surface, for example the first layer can include a first outside surface and a first inside surface, the second layer can include a second outside surface and a second inside surface, and the third layer can include a third outside surface and a third inside surface. Thus in forming the stacked layer, for example, the inner surface of the first layer will be proximate to the outer surface of the second layer. Further, in some embodiments in accordance with the present invention, a DUST or RUST can include one or more adhesive portions to fix proximate surfaces to one another and/or to fix the DUST or RUST to a man or boy's pants.

### BRIEF DESCRIPTION OF THE DRAWINGS

FIG. 1 depicts exemplary first, second and third layers of embodiments in accordance with the present invention.

FIG. 2 is a photograph illustrating the absorbency of a second layer, disposed over a first layer, and showing a removed third layer of an exemplary embodiment in accordance with the present invention.

FIGS. 3-11 depict various placements of an adhesive material on surfaces of the first, second and/or third layer of the stacked layer having a cross-shaped opening formed therein in accordance with embodiments of the present invention.

FIG. 12 is a photograph of a DUST illustrating further possible locations for the adhesive material, in accordance with embodiments of the present invention.

FIG. 13 depicts an embodiment in accordance with the present invention where a string mechanism is used to secure A DUST or RUST to a user.

FIG. 14 depicts a DUST having a cut portion rather than a cross-shaped opening, in accordance with some embodiments of the present invention.

FIG. 15 is a photograph of a DUST roll in accordance with some embodiments of the present invention.

### DETAILED DESCRIPTION

Embodiments in accordance with the present invention are now described with reference to the drawings. For purposes of explanation, numerous specific details of such embodiments are set forth in order to provide a thorough understanding thereof. While it may be evident to a skilled artisan that aspects of such embodiments can be combined in configurations not specifically described herein, such alternative configurations that rely upon the teachings of the present invention are considered to be within the scope of the present invention and the claims presented herewith.

Disclosed herein is a disposable urine shield towel (DUST) and a reusable urine shield towel (RUST) for men and boys. The various embodiments of such a DUST and/or RUST can be utilized to prevent drops of urine and/or waste water from soiling clothing. For example, such embodiments can be utilized by boys while learning hygienic urination, for example in a urinal found in a public restroom.

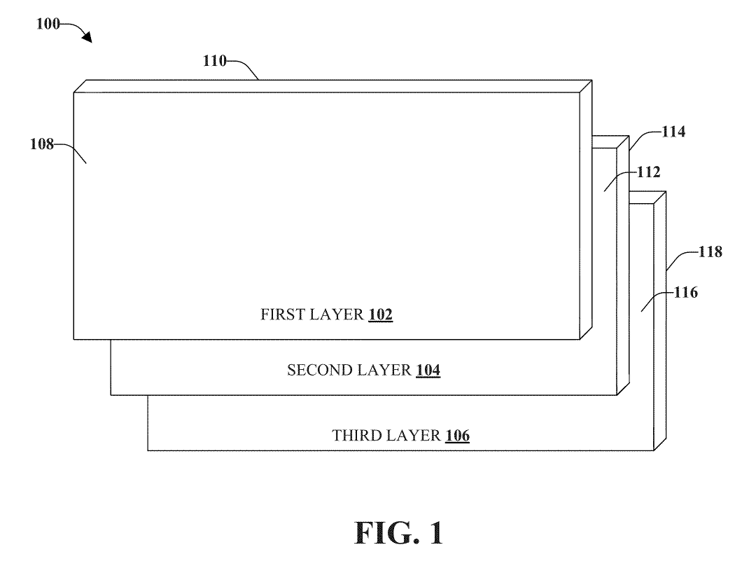

Referring initially to FIG. 1, disposable urine shield towel or DUST 100 can be utilized during urination to prevent splash back from a urinal from soiling clothing or a person. DUST 100 can be suitable for use by any male, both adult and children. For example, the DUST can be utilized by men and/or boys and can be configured to keep an upper side of pants, underwear and/or the person himself clean. According to some embodiments the DUST can be dispensed in public restrooms. According to other embodiments, the DUST can be packaged for private in-home use or be a reusable urine shield towel (RUST).

The DUST 100 can include a stacked layer. Included in the stacked layer can be a first layer 102, a second layer 104, and a third layer 106. According to some embodiments, fewer than three layers can be utilized. However, in accordance with other embodiments, more than three layers can be utilized. Generally, each layer of the stacked layers is a thin, flexible layer.

First layer 102 can be formed of an absorbent material. Examples of materials that can be used to form first layer 102 include, but are not limited to, cellulosic or fiber-based material (e.g., cotton, fluff pulp, paper, sponge, tissue), superabsorbent polymers, cloth, synthetic disposable materials, or reusable materials.

According to some embodiments, first layer 102 is formed of layers of fabric or other materials. For example, one or more layers derived from bamboo, cotton, hemp, microfiber, and so forth. Multiple layers (a layered construction) can allow transfer and distribution of urine from an outside of first layer 102 toward second layer 104 and/or third layer 106. As used in this context, the term “outside” indicates the portion away from the body, wherein “inside” refers to the portion near the body. (e.g., the outside of a pair of pants is exposed to (and seen) by others, and the inside of a pair of pants is the portion not seen when the pants are being worn).

According to some embodiments of the present invention, first layer 102 can be configured to absorb as much liquid as possible, wherein second layer 104 and third layer 106 can be utilized as additional shields to protect the user, the user's pants, and/or the user's underwear or other clothing from urine and/or waste water contamination. For example, urine drops and/or back splash from a urinal (or other place upon which someone is urinating or spraying liquid) can be large or so numerous that first layer 102 may not be able to absorb (e.g., hold) all the liquid. Therefore, the liquid can be transferred, or soaked through to second layer 104 and/or third layer 106.

Second layer 104 can be formed, for example, of a plastic material or a plastic-type material. According to some embodiments of the present invention second layer 104 can be formed of a fabric that naturally repels liquid and/or that has been treated to repel liquid. The fabric of layer 104 can be formed of a natural fabric, a synthetic fabric, or combinations thereof. According to some embodiments, second layer 104 can be laminated or coated with a waterproof material. Examples, of waterproof material include, but are not limited to, fluoropolymers, polyurethane (PU), polyvinyl chloride (PVC), rubber, wax, and the like. For example, second layer 104 can be formed of the same or a similar material as first layer 102 and/or third layer 106 where such material is coated or otherwise treated to exhibit waterproof qualities.

In some embodiments in accordance with the present invention, second layer 104 can be larger than first layer 102 and third layer 106. For example, second layer 104 can extend outward from the DUST 100 further than both first layer 102 and third layer 106.

Third layer 106 can be formed of the same or similar material as first layer 102. For example, first layer 102 and third layer 106 can be formed of absorbent material. However, the disclosed aspects are not limited to this embodiment and first layer 102 and third layer 106 can be formed of different materials. Further, second layer 104 can also be formed of a material that is similar to, the same as, or different from first layer 102 and/or third layer 106 with the proviso that such second layer 104 is coated or treated to repel or be non-absorbent to urine and/or water.

For example, first layer 102 can be formed of a first material and second layer 104 can be formed of a second material, different from the first material. Further to this example, third layer 106 can be formed of a third material, different from both the first material and the second material.

In accordance with another feature of embodiments in accordance with the present invention, first layer 102 and third layer 106 can be formed of a first material. Further, second layer 104 can be formed of a second material, different from the first material.

According to some embodiments of the present invention, first layer 102, second layer 104, and third layer 106 can be formed of materials that can be washed and reused multiple times to form a reusable urine shield towel (RUST). For other embodiments in accordance with the present invention such layers are not reusable and thus such embodiments form a disposable urine shield towel (DUST).

Even though first layer 102 and third layer 106 have been discussed as being formed of absorbent material, heavy urine drops can wet the material on the outside (e.g., side located away from the body or towards the urinal) of such layers. Therefore, second layer 104 can operate as a shield to further protect the user, the pants, and/or the underwear. For example, third layer 106 can be the outermost layer (e.g., the layer closest the urinal) and first layer 102 can be the layer closest to the person. In this case, third layer 106 might become soaked, at least in some areas, and second layer 104 and first layer 102 operate as a further barrier of liquid penetration through all the layers.

As illustrated and described, second layer 104 can be sandwiched between first layer 102 and third layer 106. For example, to form a DUST (or RUST) 100, second layer 104 can be placed over first layer, and third layer can be placed over second layer 104.

In further detail, first layer 102 can include a first outside surface 108 and a first inside surface 110. Second layer 104 can include a second outside surface 112 and a second inside surface 114. Third layer 106 can include a third outside surface 116 and a third inside surface 118.

Thus to construct DUST or RUST 100, first inside surface 110 of first layer 102 can be placed in contact with second outside surface 112 of second layer 104. Further, second inside surface 114 of second layer 104 can be placed in contact with third outside surface 116 of third layer 106. This process can be repeated for additional layers for towels that include more than three layers.

In some embodiments, the outside surface and the inside surface of each respective layer are formed of the same or of similar materials, as will be described in further detail below. However, in other embodiments, the outside surface and/or the inside surface might be formed of a different sub-layer of material and/or might be coated or sprayed with liquid resistant material. For example, an outside surface might be sprayed, while an inside surface is not sprayed.

According to some embodiments, first layer 102 and third layer 106 (and in some embodiments second layer 104) can be formed of sub-layers of fabric or other materials. For example, one or more sub-layers can include bamboo, cotton, hemp, microfiber, and so forth. Sub-layers (e.g. wherein each layer is formed via a layered construction) can allow transfer and distribution of urine or other liquids (or a semi-liquid composition) from an outside of first layer 102 (or third layer 106) toward second layer 104. However, according to some embodiments, first layer 102 (or third layer 106) can be configured to absorb as much liquid as possible, wherein second layer 104 can be utilized as an additional shield to protect the pants, underwear, skin, and so forth.

For some embodiments in accordance with the present invention, first layer 102, second layer 104, and third layer 106 can be formed of materials that can be washed and reused multiple times thus forming a reusable towel.

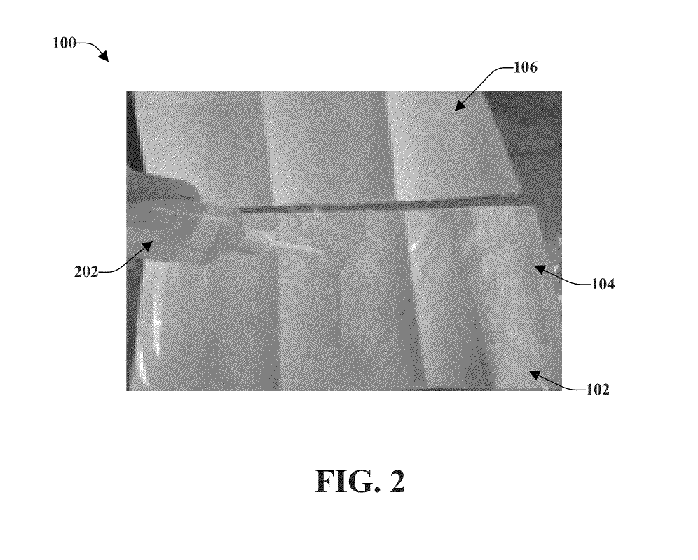

FIG. 2 is a photograph of second layer 104, placed over first layer 102 where third layer 106 has not yet been placed on second layer 104. Also shown in the photograph is water bottle 202, demonstrating that when liquid droplets are dispensed from water bottle 202, the water resistant features of second layer 104 mitigate the amount of liquid that penetrates through second layer 104 to first layer 102.

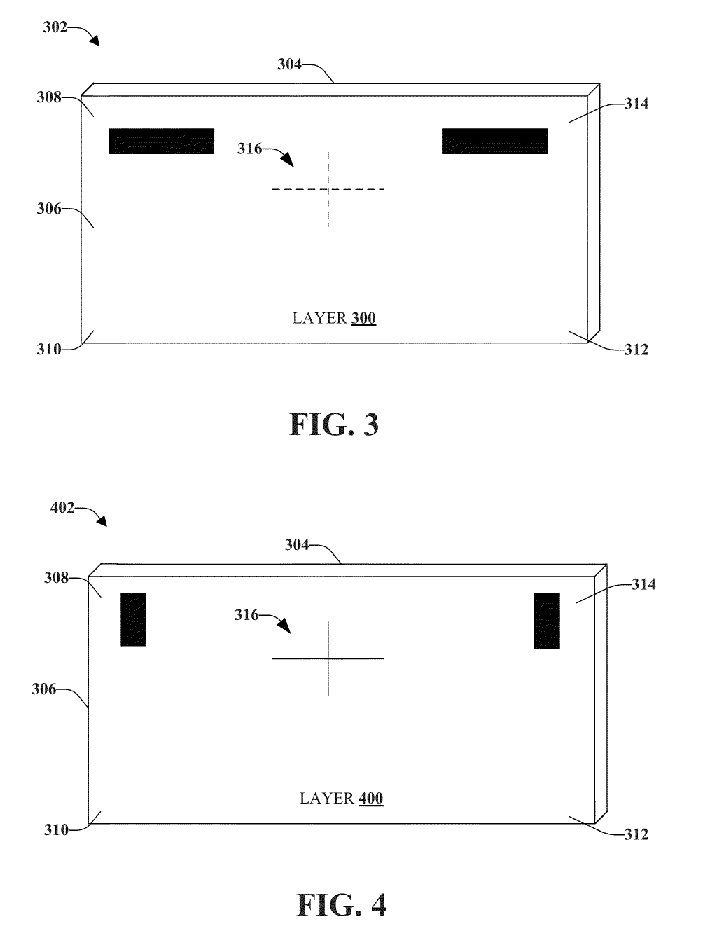

FIGS. 3-11 respectively illustrate an exemplary layer 300 of a disposable napkin 302. Layer 300 can be first layer 102 or third layer 106 (of FIG. 1) and thus serves to represent, in alternate embodiments, both first layer 102 and third layer 106 (of FIG. 1.), as will be now described.

Layers 300-1100 shown in each of FIGS. 3-11, respectively, can include an outside surface 304 (e.g., the surface located away from the body) and an inside surface 306 (e.g., the surface intended to be located nearest the body). One or more corners of the inside surface 306 can include adhesive material. For example, adhesive material might be placed on first layer 102 (of FIG. 1), on third layer 106 (of FIG. 1), or on both first layer 102 and third layer 106. The adhesive material can be utilized for securing the disposable napkin to the pants or other area (e.g., underwear, skin, and so on). According to some aspects, the adhesive material can be utilized for adjustment of the napkin to the pants.

The adhesive material can be any substance that can be applied to the surface of one or more layers. For example, the adhesive material can comprise resealable tapes, which might be adhesive or Velcro tapes, according to some aspects.

The adhesive material should bind to the one or more layers and be configured to bind, at least temporarily, to an article of clothing (e.g., a pair of pants). Further, the adhesive material should allow the disposable napkin to be removed from its positioning on the pair of pants, for example, without leaving residue or marks. Use of an adhesive material helps to prevent the disposable towel from moving away from the pants during use.

For example, adhesive material can be formed (or placed), at a first corner 308, a second corner 310, a third corner 312, and/or a fourth corner 314. The first corner 308 and fourth corner 314 can represent an upper or top portion of the disposable napkin 302 and the second corner 310 and third corner 312 can represent a bottom or lower portion of the disposable napkin 302.

According to some embodiments, for example as depicted in FIG. 3, an adhesive material might be placed near the first corner 308 and the fourth corner 314. For example, adhesive material might be placed as indicated by the block boxes.

FIG. 4 depicts an alternate placement of adhesive material located on layer 400. In this example, the adhesive material is oriented on the outside layer differently than the orientation of the adhesive material illustrated in FIG. 3.

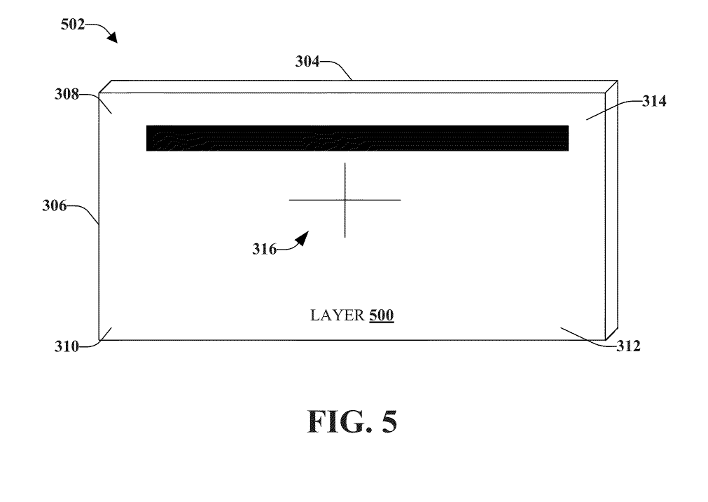

FIG. 5 shows another alternate placement of adhesive material located on layer 500 of a disposable napkin 502. In this figure the adhesive material is located at a top portion of the outside layer.

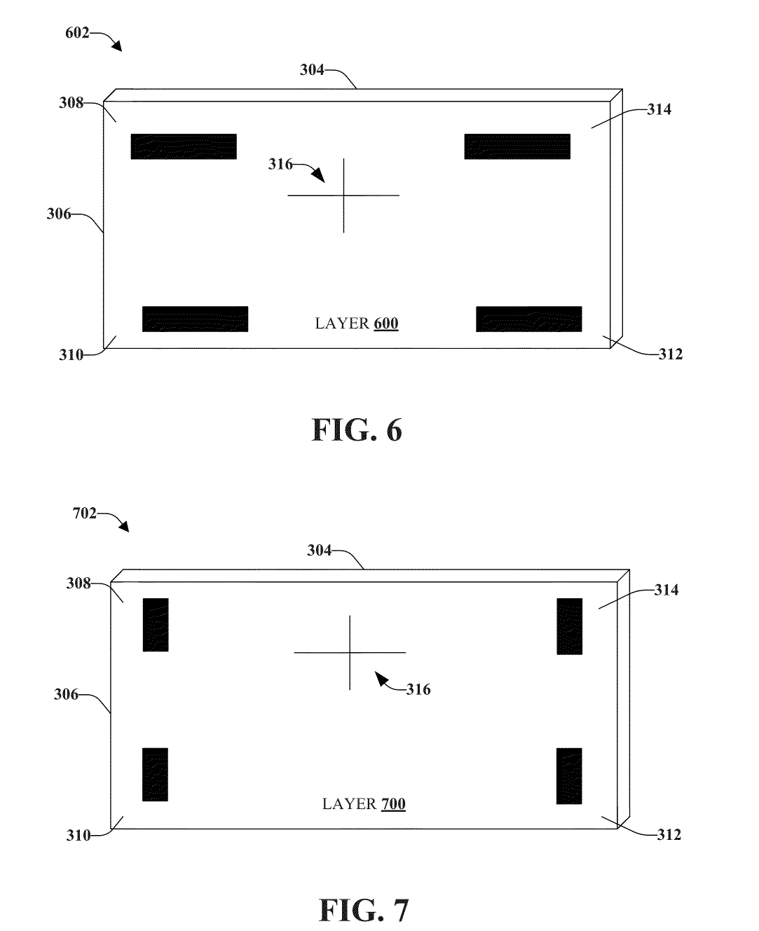

FIG. 6 illustrates another alternate placement adhesive material located at the corners of a layer 600 of a disposable napkin 602. Here, the adhesive material is placed at both a top portion and a bottom portion of the towel. Thus the adhesive material is placed near the first corner 308, the second corner 310, the third corner 312, and the fourth corner 314 having a long axis thereof in a horizontal orientation.

FIG. 7 depicts yet another alternate placement of the adhesive material. This alternate is similar to that of FIG. 6, differing only in that the long axis of the adhesive material has a vertical orientation.

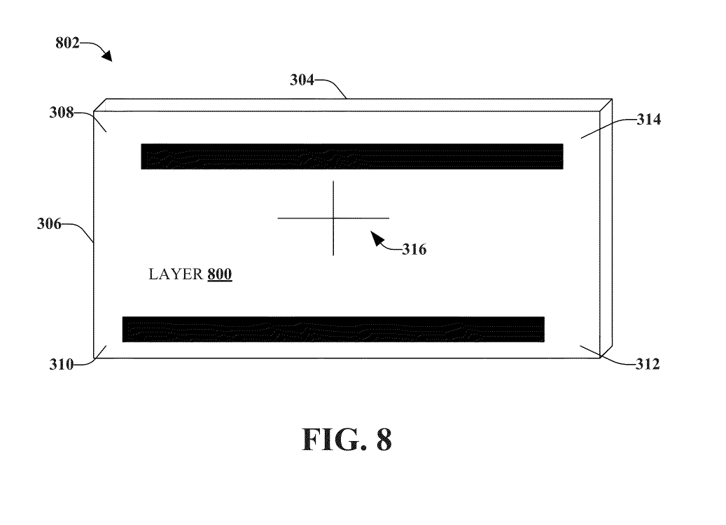

Referring now to FIG. 8 still another alternate placement of adhesive material in accordance with embodiments of the present invention is shown. As depicted, the adhesive material is in a strip form located at both a bottom portion and a top portion of DUST/RUST 802. In some embodiments, the adhesive material can be located at only the top portion or at only the bottom portion of the layer 800. In yet other embodiments, the adhesive material might be located at the top (or bottom) and one (or both) sides of the layer 800 or at the top, bottom, and one (or both) sides of the layer 800.

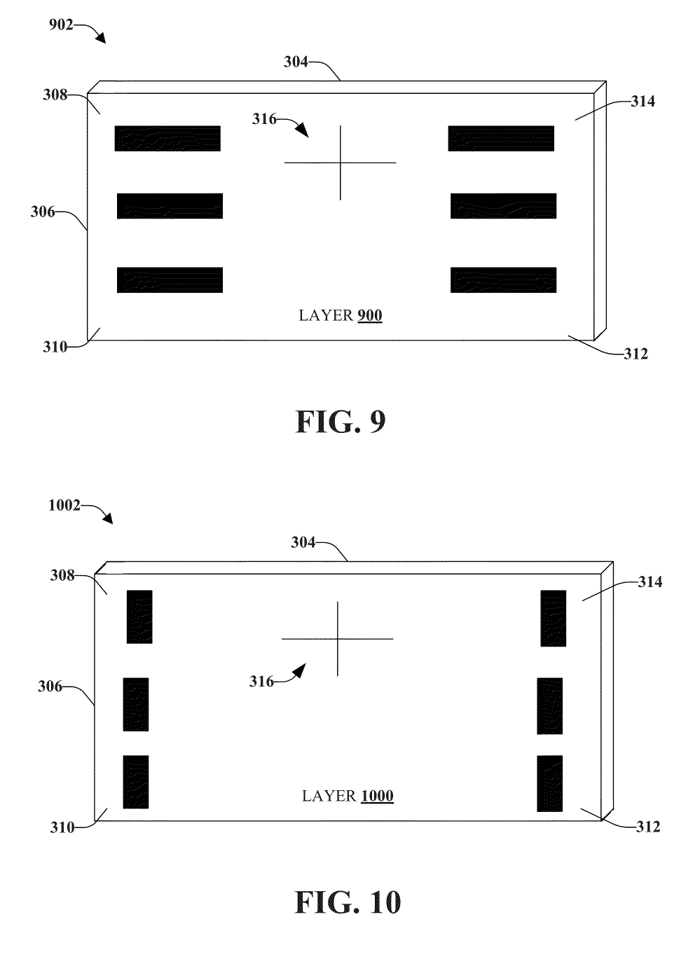

FIG. 9 depicts a further exemplary placement of the adhesive material, here on layer 900 of towel 902. As shown, such placement includes multiple portions of adhesive material.

In FIG. 10, a placement of adhesive material similar to that shown in FIG. 9 is depicted where on the orientation of a long axis of the adhesive material has been rotated 90 degrees.

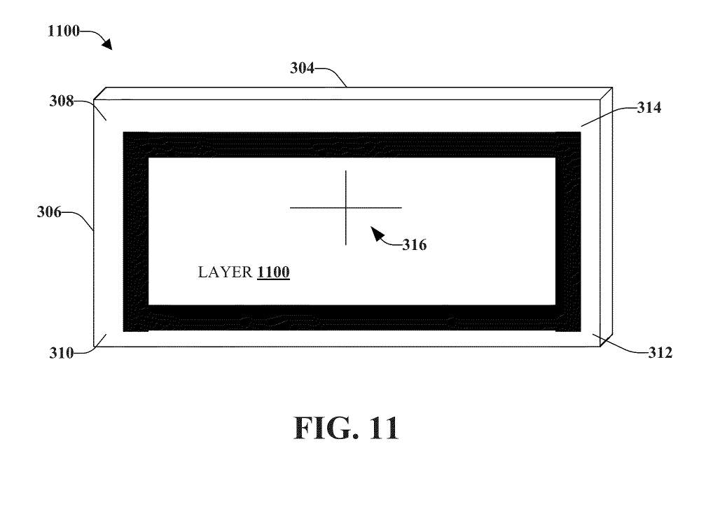

FIG. 11 depicts layer 1100 of a towel 1102 that has a perimeter of adhesive material. The adhesive material is shown having a continuous rectangular orientation. However, it should be understood that rather that a continuous rectangular shape or orientation, the adhesive material can be distinct pieces thereof so as to not form an unbroken perimeter.

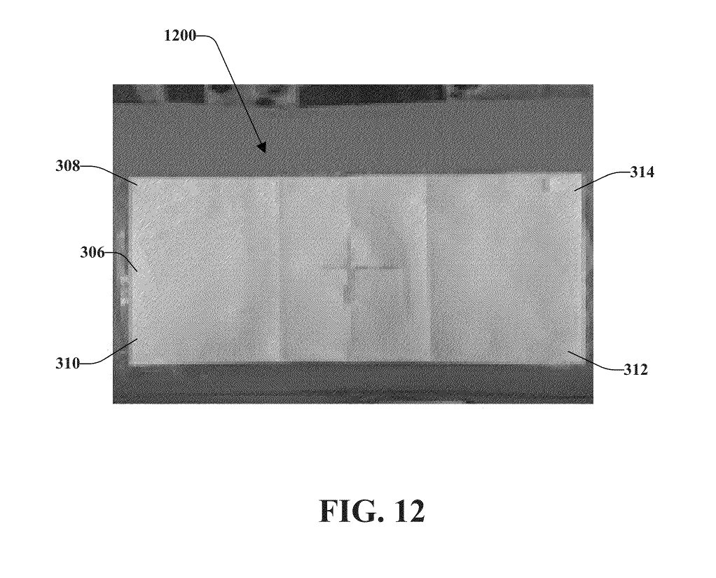

FIG. 12 is a photograph of towel 1200 depicting yet further possible locations for the adhesive material. In this photo, the disposable napkin 1200 is shown placed on a board.

Although the adhesive material is shown being placed at various particular locations, the embodiments in accordance with the present invention are not limited to these specific locations. Instead, any number of possible configurations, orientations, and/or placements can be utilized.

As further shown in FIGS. 3-12, men or boys can use the DUST/RUST by putting their penis through cross-shaped slit 316 to allow for urination. Cross-shaped slit 316 can be located near an upper side of the disposable towel and, according to this configuration, the user does not have to hold the towel while urinating. Rather the towel is held in place by the penis inserted through cross-shaped slit 316. Alternately, some embodiments in accordance with the present invention have a hole formed through the stacked layer rather than a cross-shaped slit.

The cross-shaped slit 316 can have raw edges that are not reinforced after the opening is made. Therefore, in some embodiments, the edges of the cross-shaped slit 316 are reinforced, such as by stitching, hemming, hot glue, or through other reinforcement techniques.

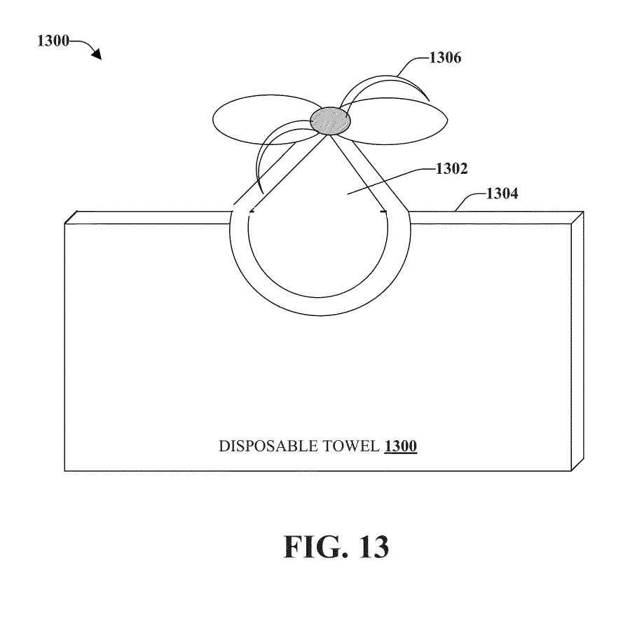

FIG. 13 illustrates another example, non-limiting disposable towel 1300 that utilizes a string mechanism to secure the disposable towel in position, according to an implementation. For example, an opening 1302 can be located at the top edge 1304 of the disposable towel 1300. The user can place his penis in the opening and secure the disposable towel 1300 by tying the strings 1306. The disposable towel 1300 can also include adhesives, as described above.

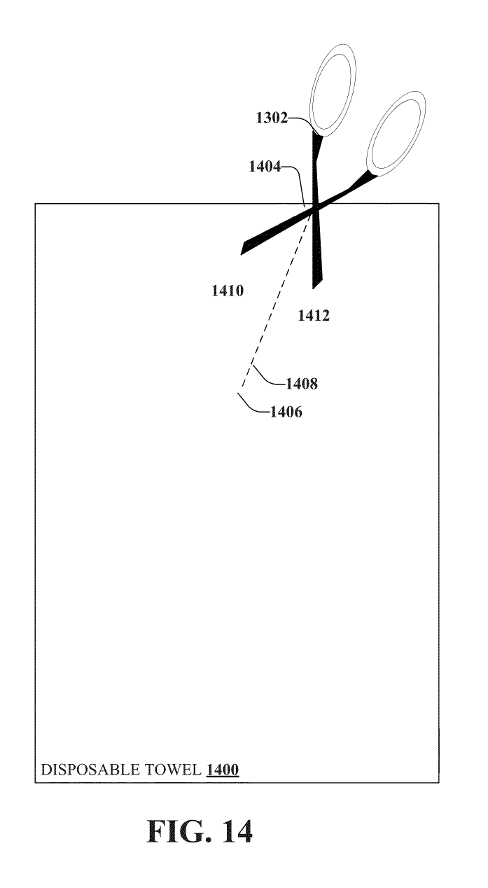

FIG. 14 illustrates another example, non-limiting towel 1400 that includes a cut portion, according to an aspect. According to this implementation, a slit or cut is provided from an upper portion of the towel (rather than the opening 1302 of FIG. 13) and continues to an outside of the towel. The slit can be created in the towel at about the same time as the layers are secured together. The slit can be made similar to taking a pair of scissors 1402 and cutting the towel from a top edge 1404 to a portion 1406 located on the upper side of the disposable towel. An example cut line is illustrated by the dotted line 1408. In this implementation, the penis can be placed in the cut portion of the towel. The sides of the cut portion, illustrated as a first side 1410 and a second side 1412 can be used to wrap the towel around the penis. The towel 1400 can also include adhesives, as described above. The edges of the cut portion can be unreinforced, or can be reinforced.

It is also noted that although the disposable towels (also referred to as a towel, napkin, disposable napkin, urine towel, or similar terms) are illustrated as a rectangular towel that has a certain width and length, the disclosed aspects are not limited to these embodiments. Instead, the disposable napkin might be longer than it is wide. In another example, the disposable napkin might be formed as a square, a triangle, or another configuration. The selection of size, length, width, and so on can be based on various criteria including prevention of urine drops and drops of mixed urine and dirty water that splash back from the urinal from soiling clothing or the user.

According to some implementation, a urine shield towel for children can be smaller. Further, the urine towel for children can have images printed thereon. For example, cartoon characters, themes, words, symbols, or other items can be printed thereon.

As discussed herein, a DUST or RUST, which has an opening, can be used by adjusting the towel such that the towel hangs from the penis and covers a large area of an upper part of the pants. In this manner, any unwanted splash that includes liquid spray and/or drops will fall onto the towel, leaving the pants clean and dry. As discussed herein, the towel is absorbent and either disposable or reusable, a DUST or a RUST.

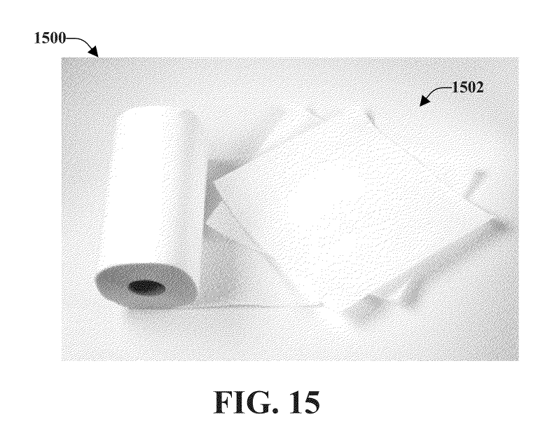

FIG. 15 depicts an exemplary towel roll 1500, in accordance with some embodiments of the present invention. Disposable towel roll 1500 can allow for a number of individual towels 1502 (two of which are illustrated) to be rolled and maintained in a dispenser. During manufacture, the disposable towels can be formed of sheets that comprise the layers as discussed herein wherein individual towels can be partially connected, for example by perforations, to allow a user to remove a single urine shield towel from the roll. A dispenser for such a roll can keep the roll stored therein clean and dry.

The subject matter as described above includes various exemplary embodiments and features thereof. However, it should be appreciated that since it is not possible to describe every conceivable component or methodology in the descriptions thereof, a skilled artisan may recognize that further combinations or permutations may be possible. In addition, various methods or constructs may be employed to implement the subject invention, modifications, variations, or equivalents thereof. Accordingly, all such embodiments of the aspects described herein are within the scope of the claims included herewith.

In view of the exemplary systems shown and described herein, methodologies for manufacturing, assembling, and using a DUST and/or RUST can be implemented in accordance with the disclosed subject matter.

What is claimed is:

1. A urine shield towel, comprising:
a stacked layer comprising a first layer, a second layer, and a third layer, wherein the second layer is sandwiched between the first layer and the third layer; and
an opening through an upper portion of the stacked layer.

2. The urine shield towel of claim 1, wherein the first layer and the third layer comprise a material that absorbs urine and/or water.

3. The urine shield towel of claim 1, wherein the second layer comprises a material that is non-absorbent with respect to urine and/or water.

4. The urine shield towel of claim 1, wherein the second layer comprises a natural fabric.

5. The urine shield towel of claim 1, wherein the second layer comprises a synthetic fabric.

6. The urine shield towel of claim 1, wherein the first layer comprises a first outside surface and a first inside surface, the second layer comprises a second outside surface and a second inside surface, and the third layer comprises a third outside surface and a third inside surface.

7. The urine shield towel of claim 6, wherein the first inside surface contacts the second outside surface, and the second inside surface contacts the third outside surface.

8. The urine shield towel of claim 6, wherein the first layer and the third layer each comprise a material that absorbs urine and/or water.

9. The urine shield towel of claim 6, wherein the second layer comprises a material that is non-absorbent with respect to urine and/or water.

10. The urine shield towel of claim 6, wherein the second layer comprises a natural fabric.

11. The urine shield towel of claim 6, wherein the second layer comprises a synthetic fabric.

12. The urine shield towel of claim 1, further comprising one or more adhesive portions.

13. The urine shield towel of claim 1 wherein such towel is disposable.

14. The urine shield towel of claim 1 wherein such towel is reusable.

15. The urine shield towel of claim 1 wherein the opening is a cross-shaped opening.

16. The urine shield towel of claim 1 wherein the opening is a cut portion of the towel.

17. The urine shield towel of claim 1 wherein the first layer comprises a cellulosic or a fiber-based material.

18. The urine shield towel of claim 1 wherein the third layer comprises a cellulosic or a fiber-based material.

19. The urine shield towel of claim 4 wherein the second layer is laminated or coated with a waterproof material.

20. The urine shield towel of claim 19 wherein the waterproof material is selected from the group consisting of fluoropolymers, polyurethane (PU), polyvinyl chloride (PVC), rubber, or wax.
